(19) United States
(12) Patent Application Publication
(10) Pub. No.: US 2024/0131213 A1
(43) Pub. Date: Apr. 25, 2024

(54) Device and Method for Dispensing Volatile Substances

(71) Applicant: CTR, LDA, Samora Correia (PT)

(72) Inventors: David Dycher, Port Erin (IM); Pedro Quieroz Vieira, Belas (PT); Teruo Hishiki, Tokyo (JP)

(21) Appl. No.: 18/548,397

(22) PCT Filed: Mar. 2, 2021
(86) PCT No.: PCT/EP2021/055201

§ 371 Date: Aug. 29, 2023

(51) Int. Cl. A61L 9/03; G01K 15/00

(52) U.S. Cl. CPC A61L 9/037; G01K 15/005; A61L 2209/111; A61L 2209/133

(57) ABSTRACT

The invention relates to a device and method for dispensing volatile substances, in particular fragrances and/or active ingredients, and comprises a housing (2), a container (5) for the substance (6) to be dispensed and a wick (18) as capillary element which is in contact with the substance (6) to be dispensed and which has a wick-side substance-dispensing region (20). According to the invention the device (1) has a temperature measuring device for direct or indirect measurement of the wick temperature (TD) at the wick-side substance-dispensing region (20), having at least one temperature sensor (13, 14) and having a measuring value output at which, during operation, an electrical wick temperature measuring signal corresponding to the currently measured wick temperature (TD) is supplied. The device (1) has furthermore an open-loop control device or a closed-loop control device for controlling the wick temperature (TD) at the wick-side substance dispensing region (20). The control device comprises a control module (30).

The invention relates to a device for dispensing, in particular for vaporising, volatile substances, in particular fragrances and/or active substances, according to the preamble of claim 1. Furthermore, the invention relates to a method for dispensing, in particular for vaporising, volatile substances, in particular fragrances and/or active substances, according to the preamble of claim 15.

Devices for dispensing, in particular for vaporising, volatile substances, in particular fragrances and/or active substances, are generally known and generally comprise a device housing and a container in which a substance to be dispensed is accommodated and which is at least partially insertable into the housing. A wick is arranged in the container as a capillary element, which projects beyond the container with a free wick end and is in contact with the substance to be dispensed in such a way that this is conveyed towards the free wick end by means of the capillary action of the wick. A heating element, in particular an electrical heating element, is regularly associated with the free wick end, by means of which heat can be applied to the free wick end in order to be able to quickly release the substance accumulating in the free wick end into the environment or to evaporate it. Such a structure is known, for example, from WO 2017/215726 A1. In addition, it is generally known to adjust the degree of evaporation and thus the evaporation power by changing the heat transfer to the free wick end. For this purpose, it is generally known to adjust the electrical power supply of the heating element, in particular in three heating stages, low, medium and high. In addition, it is known from WO 98/58692 A1 that, in order to adjust the degree of evaporation and the evaporation output, the container together with the wick can be mounted in the housing of the device in a height-adjustable manner in such a way that the relative position of the wick end to the heating device and thus the heat transfer can be changed.

The evaporation rate and thus the delivery rate of the substance to be delivered can be influenced with the known individual and manual settings described above, although the following problems are associated with this:

If the heating power is set too low, the evaporation rate and thus the evaporation performance are low, so that the desired effect is only insufficiently achieved. In order to avoid this, users of the device very often select and set a high, usually the highest possible heating power from the outset. This can have the disadvantage of drying out and dehumidifying the free wick end to an inadmissibly high degree, which can damage the capillary structure in the wick end by sticking and/or baking together. Contrary to the user's intention, the set high heating power thus considerably reduces the evaporation rate and thus the dispensing rate of the substance to be dispensed and, in extreme cases, no substance is evaporated any more and a partially still filled container has to be replaced. This negative effect can be intensified by further unfavourable boundary conditions, in particular by high ambient temperatures and low humidity.

In contrast, it is the object of the present invention to provide a device and a method for dispensing, in particular for vaporising, volatile substances, in particular fragrances and/or active substances, by means of which the substance delivery is made effective and stable, or a high degree of efficiency of the substance delivery is achieved, in particular also under changing boundary conditions.

This object is solved with the features of the independent patent claims. Advantageous embodiments thereof are the subject of the related subclaims.

According to claim 1, a device for dispensing, in particular for vaporising, volatile substances, in particular fragrances and/or active substances, is claimed, having a housing and having a container for the substance to be dispensed, which is at least partially insertable into the housing. The device further comprises a wick as a capillary element which is in contact with the substance to be dispensed, which wick is arranged at least partially in the container and forms part of the container, and which furthermore comprises a wick-side substance dispensing region. Furthermore, the device has at least one electrical heating element in the region of the wick-side substance dispensing region, by means of which a substance-enriched substance air flow can be generated by heat transfer to the substance dispensing region and flows out of the device and out of the housing.

According to the invention, the device comprises a temperature measuring device for direct or indirect measurement of the wick temperature at the wick-side substance dispensing region. This temperature measuring device has at least one temperature sensor and a measuring value output, at which an electrical wick temperature measuring signal corresponding to the currently measured wick temperature is output during operation.

Furthermore, the device has an open-loop control device or a closed-loop control device for controlling the wick temperature at the wick-side substance dispensing region, which in turn has a control module at which a wick temperature setpoint value, preferably in a functionally permissible setpoint value range without damaging the capillary structure of the wick end, can be set, preferably be preset and—fixed or variable—adjustable, by means of a setpoint value actuator and at which the current wick temperature measurement signal is input as an actual wick temperature value. By means of a comparator unit and a stored control algorithm, the heating power of the electric heating element is varied and adjusted to achieve and maintain the wick temperature setpoint value.

The invention is thus based on the knowledge gained that the capillary action of the free wick end is irreversibly damaged or, in extreme cases, destroyed if the free wick end is subjected to an impermissibly high temperature. With a setpoint adjustment in the range of a permissible wick temperature at which no damage or destruction of the capillary action at the free wick end occurs, an effective, long-term stable and uniform evaporation rate is achieved.

Direct measurement of the wick temperature at the wick-side substance delivery area is possible in principle, but technically difficult and/or cost-intensive e.g. due to limited installation space. Therefore, in the following embodiment, a suitable, indirect, simulated and very accurate measurement of the wick temperature at the wick-side substance delivery area is proposed:

For a particularly accurate measurement, it is taken into account that heat transfer to the wick-side substance delivery area for achieving and maintaining a wick temperature setpoint occurs on the one hand by thermal radiation (if necessary also by thermal conduction) from the heating element to the substance delivery area, but on the other hand also by convection of ambient air currently contained in the housing, which flows past the substance delivery area and carries thermal energy with it.

A temperature measuring device for the simulation and indirect measurement of the exact wick temperature at the wick-side substance dispensing region has at least two temperature sensors, preferably formed by thermistors. At least one temperature sensor (preferably one or two NTC temperature sensors as heating element temperature sensors) for measuring the heating element temperature is arranged on or in the heating element. In addition, at least one temperature sensor, preferably a (pre-calibrated) semiconductor temperature sensor, is arranged in the housing at a distance from the heating element as an ambient temperature sensor for measuring the ambient air temperature.

The temperature measuring device also has a function module, preferably in the form of a microcomputer unit (MCU), with a computing function and/or memory function, to which the at least two temperature sensors are connected for inputting the measured heating element temperature and the measured ambient air temperature. The high-precision wick temperature (TD) at the wick-side substance dispensing region is determined indirectly with the function module from the measured heating element temperature (TH) and the measured ambient air temperature (TU).

There is a functional relationship between the measured heating element temperature (TH) and the measured ambient air temperature (TU) to determine the wick temperature (TD):

TD=f(TH,TU)

This was determined in advance experimentally using a sample device of a device series, whereby a corresponding function is stored in each function module of the device series, in particular as an algorithm and/or as a characteristic diagram and/or as a characteristic curve. During operation of the device, a current wick temperature measurement signal is then determined and output at the function module (working as a wick temperature simulator) by evaluating the stored function, which signal can be further used as a wick temperature actual value signal.

For an often sufficiently accurate wick temperature determination, a simple linear equation can be used:

TD=f(TH,TU)=A×TH+B×TU+C,

where the quantities A, B, C can be determined experimentally in advance on at least one sample device, for example with a characteristic order of magnitude A=0.9; B=0.1; C=−10. To increase the accuracy of the wick temperature measurement, non-linear functions can also be stored in the function module.

To measure the ambient temperature in the housing, which is up to approx. 85° C. as an example, a pre-calibrated semiconductor temperature sensor available on the market with a measuring range of approx. −10 to 85° C. can be used. The operating temperature at the heating element, however, is considerably higher. For temperature measurement at the heating element, NTC temperature sensors are preferably used, which can withstand these higher temperatures, here for example with a measuring range of 0 to 160° C., without damage. However, temperature sensors, especially NTC temperature sensors, are usually individually different due to the manufacturing process and have, for example, strongly non-linear characteristic curves. For an accurate temperature measurement, the temperature sensors must therefore be calibrated. In the following, it is now indicated in general how such a calibration can be carried out relatively easily and with little effort in connection with the manufacture of the device:

First, the at least one heating element temperature sensor to be calibrated is combined with the pre-calibrated ambient temperature sensor in a calibration mode. For this purpose, the heating element is heated to several calibration temperature values which are measured with the pre-calibrated ambient temperature sensor. The corresponding electrical sensor values determined in combination are assigned to the calibration temperature values and stored in a memory of the function module, preferably with an interpolated characteristic curve.

In connection with the preferably used NTC temperature sensors, the following specific procedure results:

First, in a calibration mode, the NTC temperature sensor or sensors to be calibrated are measurably combined with the pre-calibrated semiconductor ambient temperature sensor. For this purpose, the heating element is heated to several calibration temperature values, preferably to three calibration temperature values of 30° C., 55° C. and 80° C., these temperature values being measured with the pre-calibrated semiconductor temperature sensor. The combined determined corresponding electrical NTC sensor values are assigned to the calibration temperature values and stored in a memory of the function module, preferably in an interpolated NTC characteristic curve for the entire measuring range, which is used in the heating element temperature measurement.

In principle, to achieve and maintain a wick temperature setpoint value, control without feedback is possible. For an exact keeping of the wick temperature at the substance dispensing region, a control with feedback is preferably used, which particularly preferably has a control algorithm as PID controller with a combination of a proportional behaviour, an integral behaviour and a differential behaviour. In this case, the specified wick temperature setpoint value is compared with the measured actual wick temperature value and a manipulated/actuating variable is formed on the output side according to the control algorithm and previously experimentally determined control parameters, which is fed to a controllable actuator for the heating element. In a particularly preferred embodiment, at least one thyristor in the form of a triac with phase angle control is used as the actuator in the AC supply of the electrical heating element. The actuating variable is used to vary the phase angle control of the triac and thus the heating power of the heating element in a controlled manner to achieve and maintain the wick temperature setpoint value. A typical heating power of the heating element is between 3 to 5 Watt.

The control can be operated continuously with continuous adjustment of the heating power over time. To reduce the required amount of data and computing power, the control is preferably operated discontinuously, whereby the control algorithm can be activated discontinuously at relatively short time intervals, preferably in the range of seconds, for the evaluation of determined increments of the control feedback. Such a discontinuous control leads here only to small and thus tolerable deviations, since the control action only has a very slow effect on the wick temperature.

For switching the device on and off, a usual switch or push-button can be provided on the housing. Alternatively, connection to a power source, for example plugging in and out of a socket, can be used for this purpose.

In a simple embodiment, for example, a wick temperature setpoint value can be fixed by the manufacturer. In order to increase efficient operation and for individual adaptation of the evaporation rate and/or for adaptation to differently used substances, it is proposed to make the wick temperature setpoint value variable and adjustable within a permissible range. In a simpler embodiment, this can be carried out by means of a manually operable setting device attached to the housing, preferably as a switch and/or push-button.

In a particularly advantageous embodiment, the device has a radio module, preferably as a WiFi module, for a bidirectional, wireless data connection between the device and an external display and control device, preferably a smartphone with an associated app.

This allows a currently set wick temperature setpoint value and/or a current wick temperature actual value and/or a possibly currently set switch-on period of the device as well as possibly further information to be displayed on a screen on the display and control device.

On the other hand, the display and control unit can be used to transmit commands to the device, such as a wick temperature setpoint value adapted to a substance currently to be evaporated. This can be done, for example, by manually entering a code indicated on a substance package or a substance container, in particular a QR code, via a keyboard or by reading it by means of a scanner, i.e. transmitting it to the device.

Alternatively or additionally, switch-on times and/or further setting commands can be given to the device with a timer function. This enables convenient handling of the device with variable settings.

The device can have a mechanical structure known in principle, wherein the heating element is annular with a central wick-receiving opening for the wick, in which the wick projects at least partially with its substance-dispensing region, preferably without contact with a gap distance from the receiving opening, and wherein the substance-dispensing region of the wick lies in the housing in the vicinity of a substance-airflow outlet opening, from which the substance-airflow flows to the outside of the housing.

The wick-side substance dispensing region is formed by a free wick end projecting beyond the container, in particular a container opening, in such a way that the heating element is associated with the wick in the region of the free wick end.

The device is preferably designed as a plug component, in particular for small component dimensions and for ease of handling, and has plug contacts projecting from the housing, which can be plugged into an electrical socket for holding and for electrical power supply, preferably with alternating voltage. An adaptation to the AC voltage and frequency available in a region of use can be set in a fixed manner, for example for 230V AC voltage and 50 Hz, or can be designed to be preselectable in a manner known per se.

Heating elements are generally known in different designs. Preferably, the heating element is intended here to be an electrical heating element which has a heating body made of a thermally conductive material and at least one electrical resistance element (for example in the form of a heating coil) which is thermally coupled to the heating body, preferably at least one electrical resistance element which is partially integrated and/or embedded in the heating body, and which can be connected to an energy source for the purpose of energy supply and/or can be supplied with electrical energy by means of an energy source.

The advantages resulting from the claimed method according to the invention correspond analogously to those of the device according to the invention, so that reference is made to the previously made explanations in order to avoid repetition.

The advantageous embodiments and further embodiments of the invention explained above and/or reproduced in the subclaims can be used individually or in any combination with one another—except, for example, in cases of clear dependencies or incompatible alternatives.

The invention and its advantageous embodiments and further embodiments are explained in more detail below with reference only to exemplary and schematic drawings.

They show:

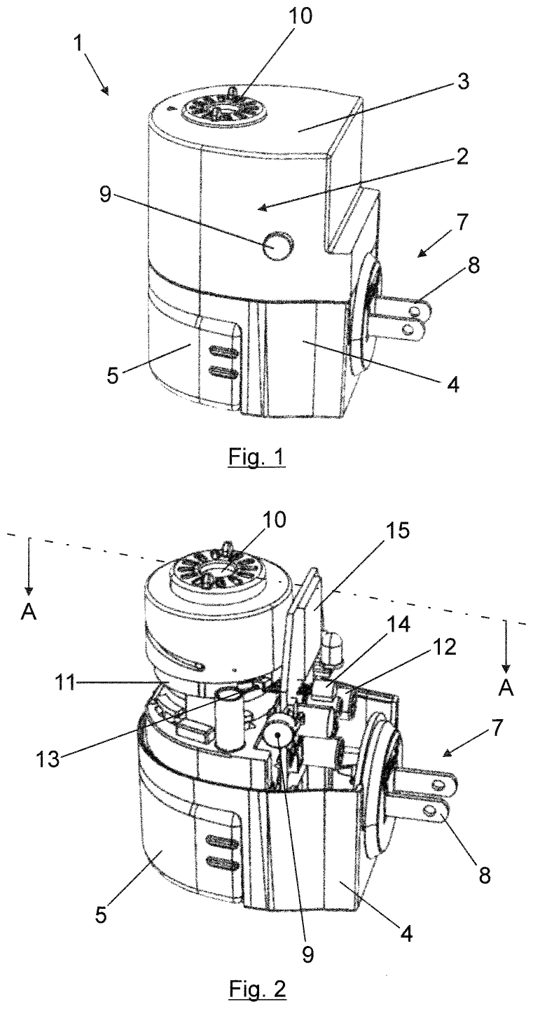

FIG. 1 shows a fully assembled evaporation device,

FIG. 2 shows the evaporation device according to FIG. 1 with the upper part of the housing removed,

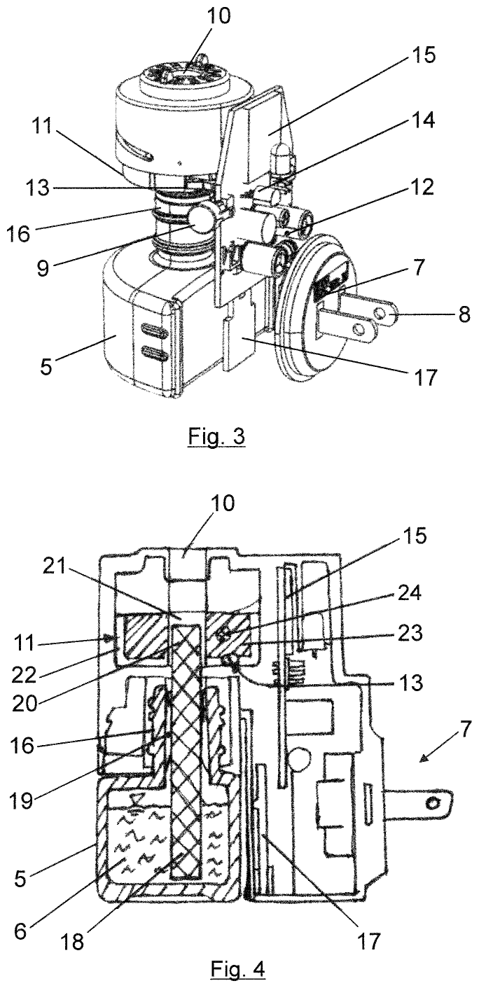

FIG. 3 the evaporation device according to FIG. 2 with the lower part of the housing removed,

FIG. 4 a longitudinal section along line A-A of FIG. 2, and

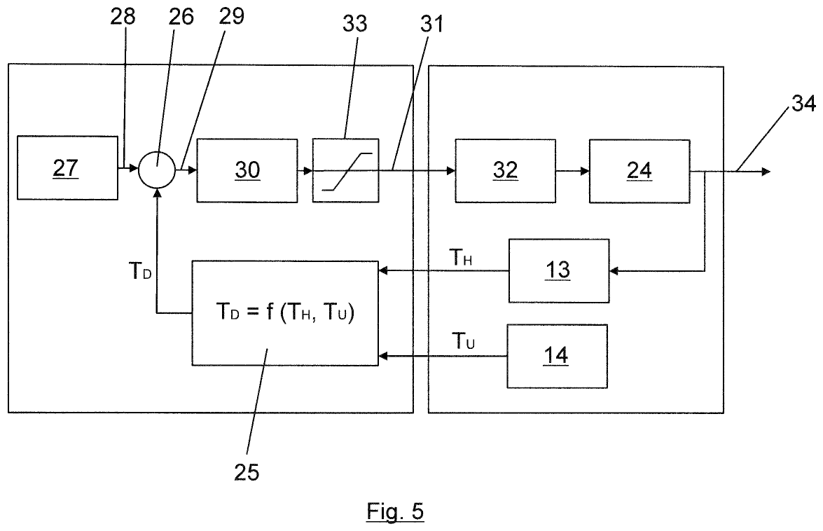

FIG. 5 a block diagram.

FIG. 1 shows an evaporation device 1 as a plug-in component which has a housing 2 formed from a housing upper part 3 and a housing lower part 4. As can be seen from FIG. 4, a container 5 is inserted into the housing 2 from below, in which a substance 6 to be vaporised is contained. Opposite the container 5 is a plug 7 with plug contacts 8 for insertion into an electrical socket, which serves here as an example as a holder for the vaporisation device 1 and for a power supply with alternating current.

In addition, an operating button 9, which can be operated from the outside, is arranged on the housing 2 for switching the vaporisation device 1 on and off and, if necessary, for selecting further functions. An exemplary outlet opening is provided on the upper side of the evaporation device 1, from which a substance air flow enriched with the evaporated substance can escape.

In FIG. 2, the upper part of the housing 3 has been removed, revealing part of the internal structure of the evaporation device 1. In the upper area, the outlet opening 10 is again visible, with an only partially visible annular heating element 11, the structure of which can be seen in FIG. 4, and the temperature TH of which is controlled by means of a controller module 12.

To determine a wick temperature TD as the actual wick temperature value, which is fed to the controller module 12, a heating element temperature sensor 13 is arranged on the heating element 11 and, at a distance from it, an ambient temperature sensor 14 is arranged in the housing 2. Also shown in FIG. 2 is the plug 7, which is electrically connected to downstream electrical components of the device 1. In addition, a radio module 15, for example in the form of a WiFi module, is mounted in the housing 2, for example for a bidirectional radio connection with a smartphone (not shown).

In FIG. 3, both the upper part of the housing 3 and the lower part of the housing 4 have been removed, showing the container 5 with its semi-cylindrical shape and its container neck 16, to which a (unspecified) level sensor 17 is assigned.

FIG. 4 shows a partially schematized cross-sectional view as a central longitudinal section along the line A-A of FIG. 2, showing that the container 5 holds both the substance to be vaporised and a wick 18 which extends upwards through the container neck 16 and a wick holder 19 and projects with a free wick end as a substance dispensing region 20 into a central wick receiving opening 21 of the ring-shaped heating element 11.

The heating element 11 has a heating element housing 22 in which an electrical resistance element 24 (shown schematically), for example a heating coil, is embedded in a thermally conductive potting material 23. The heating element temperature sensor 13 is also arranged on the heating element 11.

A presettable wick temperature as wick temperature setpoint value 38 at the free wick end 20 is controlled according to the block diagram in FIG. 5:

The heating element temperature sensor 13 detects the heating element temperature TH and the ambient air temperature TU in the housing 2 is detected by the ambient temperature sensor 14. Both temperature measurement values are supplied to a function module 25, which evaluates the two supplied temperature measurement values using a stored function to simulate and indirectly determine the wick temperature TD at the end of the wick. The wick temperature TD calculated in this way is fed to a comparator unit 26 of the control device as the current actual wick temperature value. A wick temperature setpoint value 28 set there is also fed to the comparator unit 26 as a signal from a setpoint actuator 27.

A control deviation signal 29 is then formed in the comparator unit 26, which is fed to a PID controller 30, which, in accordance with its control algorithm and stored control parameters, outputs an actuating variable 31 as an actuating signal to a heating actuator 32, which is designed here as an example as a triac with phase angle control. The actuating variable 31 is also limited here to permissible values by a signal limiter 33 to prevent inadmissible overshooting of the control. The electrical resistance element 24 of the heating element 11 is connected downstream of the actuator 32, whereby the heat output (shown schematically by arrow 34) of the heating element 11 is controlled in such a way that the wick temperature setpoint value 28 is set and maintained.

### REFERENCE LIST

1 evaporation device
2 housing
3 upper part of housing
4 lower part of housing
5 container
6 substance
7 plug
8 plug contact
9 operating button
10 outlet opening
11 heating element
12 control module
13 heating element temperature sensor
14 ambient temperature sensor
15 radio module
16 container neck
17 level sensor
18 wick
19 wick holder
20 wick end
21 wick holder opening
22 heating element housing
23 potting material
24 electrical resistance element
25 function module
26 comparator unit
27 setpoint actuator
28 wick temperature setpoint value
29 control deviation
30 PID controller
31 actuating variable
32 heating actuator
33 signal limiter
34 arrow

1. Device for dispensing, in particular for vaporizing, volatile substances, in particular fragrances and/or active ingredients, said device comprising
a housing,
a container for a substance to be dispensed, which is at least partially insertable into the housing,
a wick as capillary element which is in contact with the substance to be dispensed, and which is arranged at least partially in the container and forms part of the container, and which has a wick-side substance-dispensing region,
at least one electrical heating element in the region of the wick-side substance-dispensing region, by means of which a substance-air flow which is enriched with the substance and which flows away from the device and out of the housing by means of heat transfer to the substance-dispensing region,
wherein said device includes a temperature measuring device for measurement of the wick temperature (TD) at the wick-side substance-dispensing region, having at least one temperature sensor and having a measuring value output at which, during operation, an electrical wick temperature measuring signal corresponding to the currently measured wick temperature (TD) is supplied,
a control device for controlling the wick temperature (TD) at the wick-side substance dispensing region, and
wherein the control device comprises a control module at which a wick temperature setpoint value is set by means of a setpoint value actuator in a functionally permissible setpoint value range not damaging the capillary structure of the wick end, and at which the actual wick temperature measurement signal is input as the actual wick temperature value (TD), and which varies and adapts the heating power of the electric heating element with an algorithm by means of a comparator unit and a stored control algorithm in order to achieve and maintain the wick temperature setpoint value.

2. Device according to claim 1,
wherein the temperature measuring device for simulating and indirectly measuring the wick temperature (TD) at the wick-side substance-dispensing region has at least two temperature sensors;
wherein at least one of said temperature sensors is arranged as a heating element temperature sensor for measuring the heating element temperature (TH) on or in the heating element,
wherein at least one semiconductor temperature sensor, is arranged as an ambient temperature sensor for measuring the ambient air in the housing spaced apart from the heating element in the housing,
wherein the temperature measuring device has at least one function module as a simulation module with a computing function and/or memory function, to which the at least two temperature sensors for inputting the measured heating element temperature (TH) and the measured ambient air temperature (TU) are connected,
wherein the wick temperature (TD) at the wick-side substance dispensing region is indirectly determinable from the measured heating element temperature (TH) and the measured ambient air temperature (TU) by means of the function module in such a manner that the functional relationship between the heating element temperature (TH) and the ambient air temperature (TU) for the determination of the wick temperature (TD)
TD=f(TH,TU)
has been determined experimentally in advance on the basis of at least one sample device of a device series and a corresponding function, in particular as an algorithm and/or as a characteristic diagram and/or as a characteristic curve, is stored in each function module of the device series, and
wherein during operation of the device and by evaluating the stored function, a current wick temperature measurement signal, which is available as a wick temperature actual value signal (TD), is determined and output at or with the function module forming a wick temperature simulator.

3. Device according to claim 2, wherein the wick temperature (TD) is determined as a function of the heating element temperature (TH) and the ambient air temperature (TU) in the function module according to the equation
TD=f(TH,TU)=A*TH+B*TU+C
with the variables A, B, C being determined experimentally in advance with the at least one sample device.

4. Device according to claim 2, wherein
the heating element temperature sensor is formed by at least one NTC temperature sensor, and is calibratable, in particular during the manufacturing of the device, in such a way:
that the at least one heating element temperature sensor to be calibrated is combined in a calibration mode with the semiconductor temperature sensor as pre-calibrated ambient temperature sensor,
that the heating element is heated to a plurality of calibration temperature values which are measured with the pre-calibrated ambient temperature sensor, and
that the corresponding electrical sensor values which are determined in combination, preferably NTC sensor values, are assigned to the calibration temperature values and stored in a memory of the function module with an interpolated characteristic curve.

5. Device according to claim 1, wherein
the control module has a control algorithm in the form of a PID controller with a combination of a proportional response and an integral response and a differential response, with the predetermined wick temperature setpoint value being compared with the wick temperature actual value (TD) in a comparator unit and an actuating variable being formed on the output side in accordance with the control algorithm and regulating parameters determined experimentally in advance, and
wherein the actuating variable is fed to a controllable actuator for the heating element in such way that the actuator is at least one thyristor in the form of a triac with phase-angle control in the AC supply of the electric heating element, so that the phase-angle control of the triac and thus the heating power of the heating element is variable in a controlled manner with the actuating variable in order to achieve and maintain the wick temperature setpoint.

6. Device according to claim 5, wherein the control algorithm is activatable discontinuously at predetermined time intervals for evaluating detected increments of the feedback of the closed-loop control.

7. Device according to claim 1, wherein a manually operable setting device 7 for switching on/off and/or for setting different wick temperature setpoints values is arranged on the housing.

8. Device according to claim 1, wherein the device has a radio module 7 for a bidirectional and wireless data connection between the device and an external display and control device.

9. Device according to claim 8, wherein said display and control device displays information selected from the group consisting of a currently set wick temperature setpoint value, a current wick temperature actual value (TD), and a currently set switch-on period of the device.

10. Device according to claim 8, wherein switch-on times are presettable with a timer function and a wick temperature setpoint value adapted to a substance to be currently evaporated is settable with the display and control unit of the device via the wireless data connection in such way that a code indicated on a substance packaging or a substance container is entered manually via a keyboard or is read in by means of a scanner.

11. Device according to claim 1, wherein
the heating element is annular with a central wick-receiving opening for the wick, in which the wick projects at least partially with its substance-dispensing region, and
wherein the substance dispensing region of the wick is located in the housing in the region of a substance air flow outlet opening from which the substance air flow flows to the outside of the housing.

12. Device according to claim 11, wherein the wick-side substance dispensing region is formed by a free wick end projecting beyond the container in such a way that the heating element is assigned to the wick in the region of the free wick end.

13. Device according to claim 1, wherein the device is designed as a plug component with plug contacts projecting from the housing, which plug contacts (8) are pluggable into an electrical socket for holding and for electrical power supply.

14. Device according to claim 1, wherein the at least one heating element is an electrical heating element which has a heating body comprising a heating element housing and a thermally conductive material and at least one electrically resistive element, which is thermally coupled to the heating body, and which is providable with electrical energy by means of an energy source.

15. Method for dispensing, in particular for vaporizing, volatile substances, in particular fragrances and/or active ingredients, said method comprising the steps of:
with providing a device with a housing,
providing a container for the substance to be dispensed, which is at least partially insertable into the housing,
providing a wick as capillary element, which is in contact with the substance to be dispensed and which is arranged at least partially in the container and forms part of the container and which has a wick-side substance-dispensing region,
heating the substance-dispensing region with at least one electrical heating element to create a substance-air flow which is enriched with substance and which flows away from the device and out of the housing,
measuring the wick temperature (TD) at the wick-side substance dispensing region with a temperature measuring device by means of at least one temperature sensor, and
generating an electrical wick temperature measuring signal corresponding to the current wick temperature (TD) at a measuring value output of the temperature measuring device during operation,
controlling the wick temperature (TD) at the wick-side substance delivery region with a control device,
wherein the control device comprises a control module at which a wick temperature setpoint value is set with a setpoint value actuator, in a functionally permissible setpoint value range not damaging the capillary structure of the wick end, and at which the current wick temperature measuring signal is input as the actual wick temperature value (TD), and
varying and adapting the heating power of the electric heating element by means of a comparator unit and a stored control algorithm in order to achieve and maintain the wick temperature setpoint value.
